(19) United States
(12) Patent Application Publication
(10) Pub. No.: US 2009/0281588 A1
(43) Pub. Date: Nov. 12, 2009

(54) DETERMINING ATRIAL TIME PERIODS IN CONJUNCTION WITH REAL-TIME TESTING

(75) Inventor: Xing Pei, Thousand Oaks, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/118,423

(22) Filed: May 9, 2008

(51) Int. Cl. A61N 1/365

(52) U.S. Cl. 607/17

(57) ABSTRACT

Time periods such as PVAB and PVARP are defined based on data acquired during real-time tests. For example, data may be collected during a real-time test that determines a sensing threshold or during a real-time test that determines a capture threshold. Time period information may then be derived based on correlations between the timing of far-field events and/or retrograde conduction derived from the acquired data and the timing of sensed or paced ventricular events. In some cases, the derived time period information may be used to provide timing recommendations for initial programming of an implantable device.

### TECHNICAL FIELD

This application relates generally to implantable cardiac devices and, more specifically, but not exclusively to determining time periods that may be used for atrial sensing operations.

### BACKGROUND

An implantable medical device, such as an implantable cardiac rhythm management device (e.g., a pacemaker, a defibrillator, or a cardioverter), may be used to monitor cardiac function and provide therapy for a patient who suffers from cardiac arrhythmia. For example, in an attempt to maintain regular cardiac rhythm, an implantable device may track the type and timing of native cardiac signals. In this way, the implantable device may determine whether cardiac events (e.g., contractions) are occurring and whether they are occurring at the proper times. In the event contractions are not occurring or are occurring at undesirable times, the implantable device may stimulate the heart in an attempt to restore proper cardiac rhythm. For example, an implantable device may stimulate the cardiac muscles of one or more chambers of the heart by delivering electrical pulses via one or more leads implanted in or near the chamber(s).

The implantable device also may track cardiac signals through the use of these implanted leads. For example, the implantable device may process signals received via the leads and then attempt to characterize the received signals as a particular cardiac event. Such cardiac events may include, for example, P-waves, R-waves, and T-waves. A P-wave corresponds to a contraction (depolarization) of an atrium. A QRS complex (comprising an R-wave) corresponds to a contraction (depolarization) of a ventricle. A T-wave corresponds to a return to a resting state (repolarization) of a ventricle.

In general, an implantable device may employ some form of signal sensing circuit for cardiac event detection. For example, a sensing circuit may include a sense amplifier that includes or is associated with a signal filter. Here, the bandwidth of the filter may be selected to pass only the types of signals that the system is attempting to detect. Any signals that pass through the filter may then be provided to a threshold detector that generates an output signal in the event the amplitude of the input signal exceeds a fixed threshold level or a threshold level defined by an automatic sensitivity control scheme. The output signal may thus provide an indication of a certain cardiac event (e.g., detection of a P-wave).

By analyzing the type and timing of these indications, the implantable device may determine whether therapy should be provided and, if so, the type of therapy to be provided (e.g., stimulation pulses). For example, if the implantable device detects cardiac events at the appropriate relative times, the device may simply continue monitoring the indications. In contrast, if an indication has not been received for a defined period of time, the implantable device may deliver an appropriate stimulation (e.g., pacing) pulse to the heart. In the event too many indications are received over a given time period (e.g., a tachycardia condition is detected), the implantable device may provide a different form of therapy.

Sensing techniques such as those described above may not always provide a proper indication of cardiac events. For example, a P-wave detection circuit may indicate a detection based on a true P-wave or a retrograde P-wave in some instances while, in other instances, the circuit may improperly indicate a detection in response to reception of a far-field R-wave, a far-field T-wave, extracardiac physiologic noise, or external noise.

To address such “over-sensing,” a cardiac device may employ refractory and blanking time periods. A post-ventricular atrial blanking (“PVAB”) period may be employed, for example, to prevent over-sensing of far-field ventricles signals (e.g., R-waves and, optionally, T-waves) in the atrial channel. Here, to achieve blanking, the atrial sensing circuit may simply be turned off during the blanking period. A post-ventricular atrial refractory period (“PVARP”) also may be employed to enable analysis of a sensed signal (e.g., to identify the signal as retrograde conduction or some other type of signal) before acting on the signal. In this case, the atrial sensing circuit may remain on, yet any events that exceed a threshold during this time period may not automatically trigger an immediate response. Instead, the implantable device may process the received signal in an attempt to further characterize the signal, which may lead to a different type of response. A PVARP may be used, for example, to mitigate sensing of retrograde conducted P-waves or other signals and to improve detection of atrial fibrillation.

Under some circumstances, the use of such time periods may result in the under-sensing of certain cardiac events. For example, if an atrial event of interest (e.g., atrial fibrillation, atrial flutter, or some other event) occurs during a PVAB period, the implantable device may not detect that atrial event.

In summary, inappropriate setting of sensing time periods may result in an implantable device not providing appropriate therapy at certain times. For example, if the PVAB period is set too short upon implant of the implantable device, the implantable device may over-sense far-field ventricular signals in the atrial channel. Such over-sensing may result in, for example, incorrect diagnosis of atrial arrhythmia which may, in turn, lead to inappropriate mode switches by the implantable device. Conversely, if the PVAB period is set too long upon implant, the implantable device may under-sense atrial signals in the atrial channel. This under-sensing may result in, for example, non-detection of atrial arrhythmia which may, in turn, lead to the implantable device providing inappropriate therapy.

### SUMMARY

A summary of sample aspects of the disclosure follows. It should be understood that any reference to the term aspects herein may refer to one or more aspects of the disclosure.

The disclosure relates in some aspects to determining one or more time periods that may be employed in conjunction with cardiac therapy. Such time periods may include, for example, PVAB, PVARP, or other types of time periods that are used to detect atrial or other events.

The disclosure relates in some aspects to acquiring data during a real-time test to define an atrial time period. For example, in some cases atrial data may be collected during a real-time test relating to a sensed event (e.g., a test to determine a sensing threshold). In addition, in some cases atrial data may be collected during a real-time test relating to a paced event (e.g., a test to determine a capture threshold). In some aspects this data may be collected for a period of time defined by a detection window, where the timing of the detection window is based on the timing of ventricular depolarization (e.g., a paced or sensed ventricular event).

The disclosure relates in some aspects to defining an atrial time period based on correlations between the timing of certain atrial events derived from the acquired atrial data and the timing of sensed or paced ventricular events. In some aspects, the atrial events of interest are those that are caused by a ventricular event (e.g., a far-field or a retrograde conduction event that occurs as a result of an R-wave). Here, by collecting such event information over time, parameters such as minimum, average, and maximum timings of far-field and/or retrograde events may be derived and used to define the atrial time period.

In some implementations the defined time period may be used as a recommendation for the programming (e.g., initial programming) of an implantable device. For example, upon implant, the implantable device may determine time period information and provide this information to an external programmer that displays the information. A user of the programmer may then accept the displayed recommendation or select a time period of his or her choosing (e.g., based on a displayed range) to program the initial time periods to be used by the implantable device.

### BRIEF DESCRIPTION OF THE DRAWINGS

These and other features, aspects, and advantages will be more fully understood when considered with respect to the following detailed description, the appended claims, and the accompanying drawings, wherein:

FIG. 1 is a simplified diagram illustrating a sample implantable medical device;

FIG. 2 is a simplified flowchart of an embodiment of operations that may be performed to provide time period information for an implantable medical device;

FIG. 3 is a simplified block diagram illustrating several aspects of an embodiment of an apparatus configured to define time period information;

FIG. 4 is a simplified block diagram illustrating several aspects of an embodiment of programming apparatus;

FIG. 5 is a simplified diagram illustrating several sample fields of a display of an embodiment of a programming apparatus;

FIG. 6 is a simplified flowchart of an embodiment of operations that may be performed to defined an atrial time period;

FIG. 7 is a simplified diagram illustrating sample atrial time periods;

FIG. 8 is a simplified diagram illustrating sample atrial time periods;

FIG. 9 is a simplified diagram of an embodiment of an implantable stimulation device in electrical communication with one or more leads implanted in a patient's heart for sensing conditions in the patient, delivering therapy to the patient, or providing some combination thereof; and

FIG. 10 is a simplified functional block diagram of an embodiment of an implantable cardiac device, illustrating basic elements that may be configured to sense conditions in the patient, deliver therapy to the patient, or provide some combination thereof.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus (e.g., device) or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

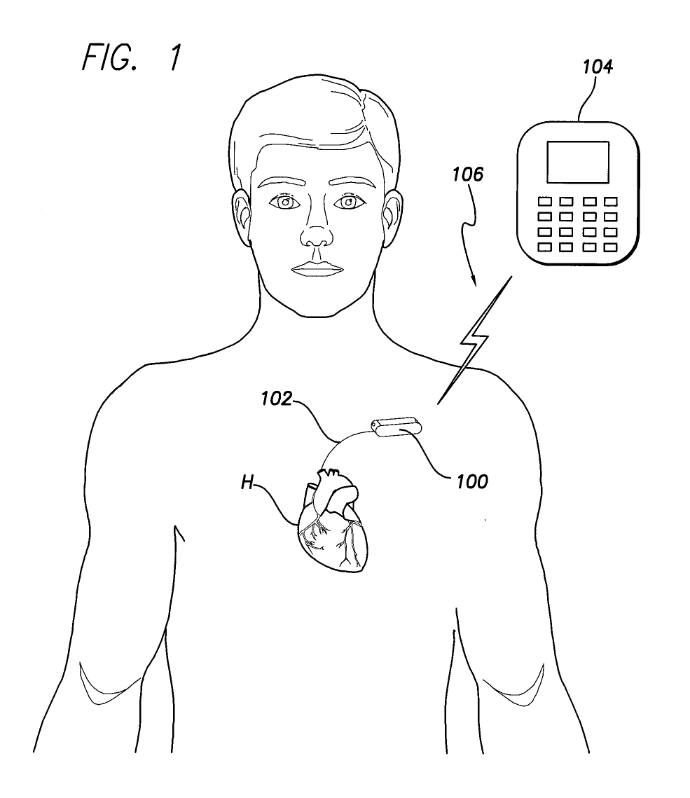

FIG. 1 illustrates, in a simplified manner, a device 100 that is implanted in a patient at a location that enables the implanted device 100 to monitor cardiac signals from and apply signals to a patient's heart H. For example, in some cases the implanted device 100 is implanted subcutaneously in the pectoral region of a patient's chest and connects to one or more implantable leads 102. Each implantable lead 102 is routed from the implanted device 100 through the patient's body and implanted within and/or on the heart H.

The implanted device 100 may be configured to communicate with an external device 104 (e.g., a programmer). For example, the implanted device 100 and the external device 104 may include respective communication components (not shown in FIG. 1) that communicate via RF signals (as represented by a symbol 106) or in some other suitable manner. Through the use of such a communication mechanism, the external device 104 may control the operation of the device 100 (e.g., by downloading operational parameters to the device 100) after implant. In addition, information acquired or generated by the device 100 after implant may be uploaded to the external device 104 via this communication mechanism.

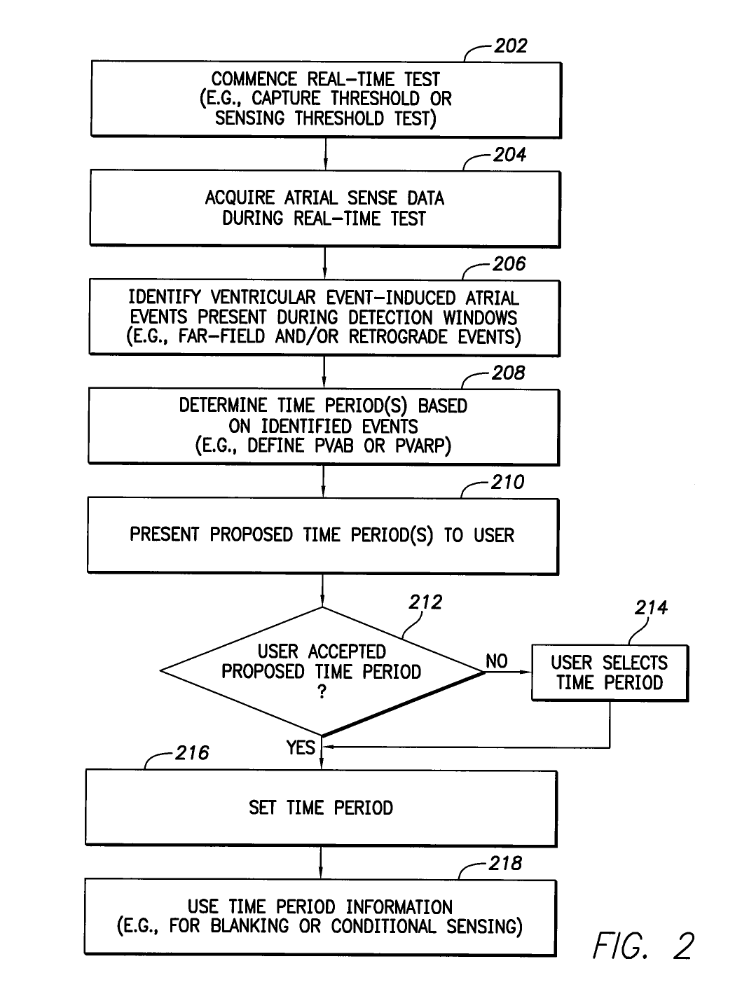

Referring now to FIG. 2, the implanted device 100 may be configured to cooperate with the external device 104 to define one or more time periods that are used in cardiac therapy-related operations. For example, the implanted device 100 may process sensed cardiac signals to determine time periods such PVAB and PVARP. The implanted device 100 may then use these time periods in conjunction with its cardiac stimulation therapy operations

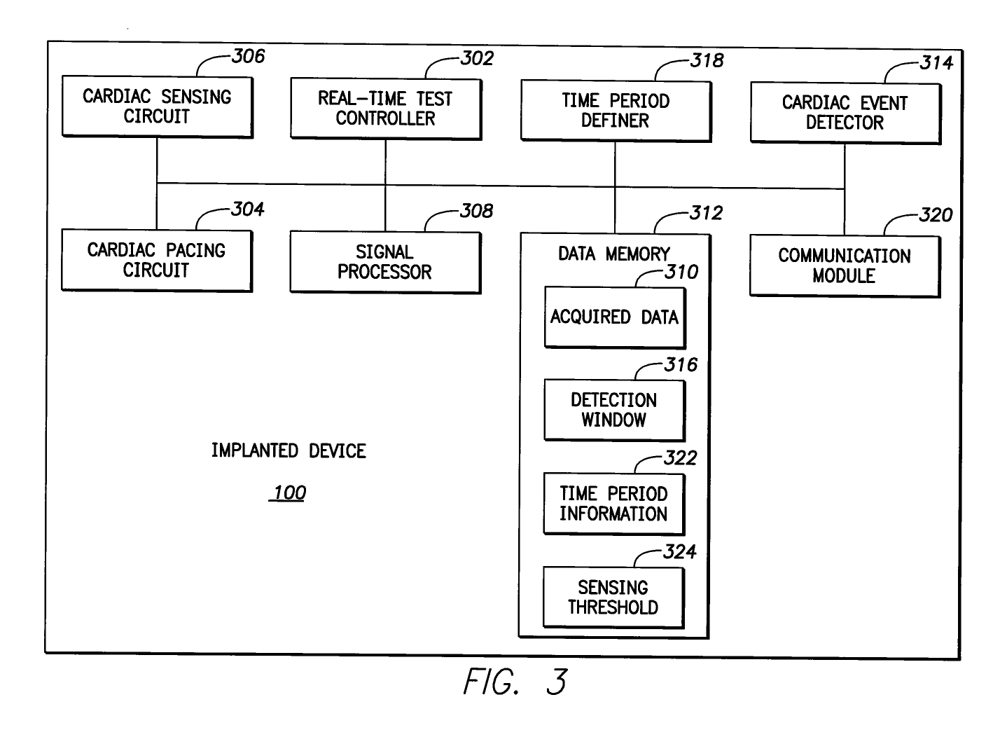

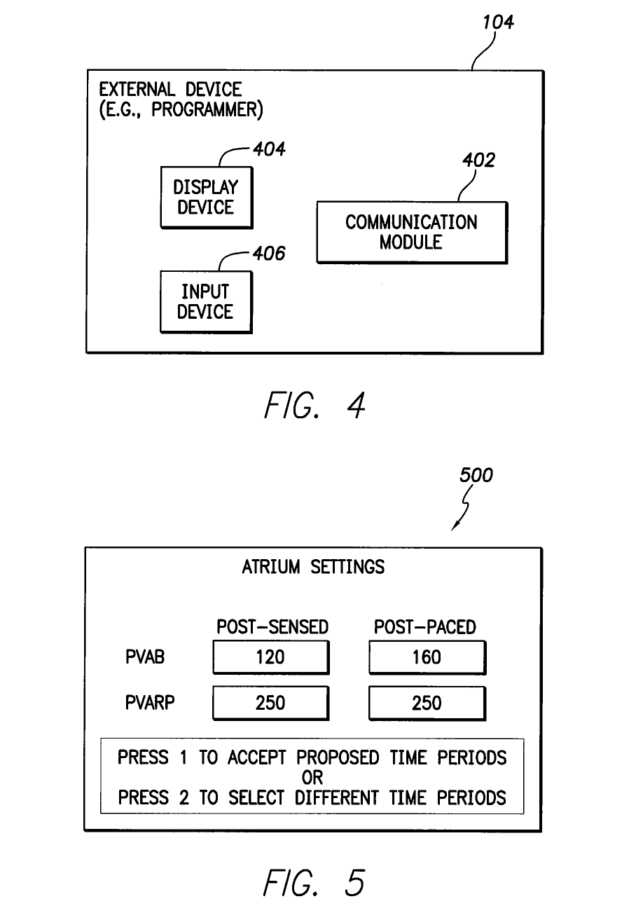

For convenience, the operations of FIG. 2 (or any other operations discussed or taught herein) may be described as being performed by specific components. For example, certain operations are described in the context of sample components of the implanted device 100 and the external device 104 as shown in FIGS. 3 and 4, respectively. It should be appreciated, however, that these operations may be performed by other types of components and may be performed using a different number of components. It also should be appreciated that one or more of the operations described herein may not be employed in a given implementation.

As represented by block 202 of FIG. 2, at certain points in time the implanted device 100 commences a test that determines one or more thresholds that are used in cardiac-related operations. For example, upon implant or during an in-clinic procedure, a real-time test controller 302 (FIG. 3) of the implanted device 100 may be configured to perform one or more real-time tests to determine a capture threshold, a sensing threshold, or both. In conjunction with these tests, a cardiac sensing circuit 306 may be configured to sense a ventricular channel. In this way, the implanted device 100 may acquire information relating to the timing of ventricular events such as ventricular depolarization (e.g., an R-wave).

As represented by block 204 of FIG. 2, the implanted device 100 also may be configured to acquire atrial sense data during a real-time test. For example, the cardiac sensing circuit 306 may be configured to sense an atrial channel during such a test.

A signal processor 308 processes the signals acquired by the cardiac sensing circuit 306 from the ventricular and atrial channels over several cardiac cycles to provide data (e.g., intracardiac electrogram data) representative of these signals. Here, the data may include information that indicates the timing relationships between events that occurred in the atrial and ventricular channels or the information may be stored in a manner that indicates these timing relationships. As indicated by the acquired data block 310 in FIG. 3, this data may be stored in a data memory 312 for use in subsequent operations.

As represented by block 206, a cardiac event detector 314 may process the data from the atrial channel to identify specific cardiac events that occur in that channel. For example, the cardiac event detector 314 may determine whether an atrial event derived from the atrial data corresponds to a far-field signal (e.g., a far-field R-wave or T-wave), a retrograde P-wave, or some other event that was induced by a ventricular event (e.g., an R-wave).

To facilitate identifying such events, the cardiac event detector 314 may be configured to identify only those events that occur during a specified interval of time. For example, a far-field R-wave and a far-field T-wave may be expected to appear in the atrial channel within a certain window of time that follows the ventricular depolarization that caused these far-field events. Similarly, retrograde conduction may be expected to appear in the atrial channel within a different window of time following ventricular depolarization and under different circumstances (e.g., upon loss of atrial capture). Such an expectation may be based on, for example, empirical evidence that shows that the corresponding cardiac timing is relatively consistent in a patient (e.g., due to a relatively consistent ventricle to atrium conduction time). Consequently, the cardiac event detector 314 may be configured to process the subset of the atrial data acquired at block 204 that corresponds to such a window of time (referred to herein as a detection window). As represented by a detection window block 316 shown in FIG. 3, this window definition information may be maintained in the data memory 312.

The cardiac event detector 314 may process data associated with several cardiac cycles to provide a set of event data representative of the far-field and/or retrograde events that occurred during these cycles. This event data may include information relating to one or more of, for example, the timing of each event, the amplitude of each event, or some other characteristic(s) of each event.

As represented by block 208, a time period definer 318 defines a time period based on the events detected at block 206. For example, the time period definer 318 may define a post-ventricular atrial time period such as PVAB based on the time difference between ventricular depolarization and a corresponding far-field event for each cardiac cycle. Similarly, the time period definer 318 may define a post-ventricular atrial time period such as PVARP based on the time difference between ventricular depolarization and a corresponding retrograde event for each cardiac cycle. Here, each defined time period may be set equal to the measured time difference plus a safety margin.

As represented by block 210, the time period information determined at block 208 may be presented to a user as one or more proposed time periods. For example, a communication module 320 of the implanted device 100 may transmit the time period information to a communication module 402 (FIG. 4) of the external device 104. The external device 104 may then output an indication of this time period information to the user (e.g., via a display device 404 or some other suitable output device).

FIG. 5 illustrates a simplified example of a display 500 that may be provided by the display device 404. In this example, the display 500 presents several atrium settings relating to PVAB and PVARP. Specifically, post-sensed and post-paced PVAB settings of 120 ms and 160 ms, respectively, are proposed. In addition, post-sensed and post-paced PVARP settings of 250 ms are proposed. The user also may be instructed to either accept these proposed settings or selects his or her own settings. It should be appreciated that time period settings may be proposed in various other ways. For example, in some cases, the display 500 may provide a range of recommended values (e.g., based on minimum and maximum time period values determined over a number of cardiac cycles).

As represented by block 212, in response to the proposed timing information presented by the external device 104, the user (e.g., a treating physician or clinician) may use an input device 406 (e.g., a keyboard, a pointing device, or some other suitable device) of the external device 104 to specify the actual time period(s) to be used by the implanted device 100. For example, at block 212 the user may simply accept a proposed time period by entering an appropriate response via the input device 406. The operational flow of FIG. 2 then proceeds to block 216 in this case.

Alternatively, as represented by block 214, the user may use the input device 406 to enter in a different time period. In this case, the user's decision may be based, in part, on the proposed time period information (e.g., in the form of a displayed range of time period values). Here, the time period information may include a safety margin or such a margin may be added to the time period information (e.g., by the user, the device 100, or the device 104).

As represented by block 216, the implanted device 100 may then specify the final time period value based on an indication received from the user input device 406. Here, after receiving an indication (e.g., a specified value) from the input device 406, the communication module 402 may transmit the indication to the communication module 320. The device 100 (e.g., the time period definer 318) may then store time period information 322 that is based on this indication in the data memory 312.

As represented by block 218, the implanted device 100 may then use the time period information 322 in subsequent operations. For example, the implanted device 100 may use a PVAB parameter to disable sensing of the atrial channel for a certain period of time after detection of an R-wave. In addition, the implanted device 100 may use a PVARP parameter to conditionally sense in the atrial channel for a certain period of time after PVAB ends.

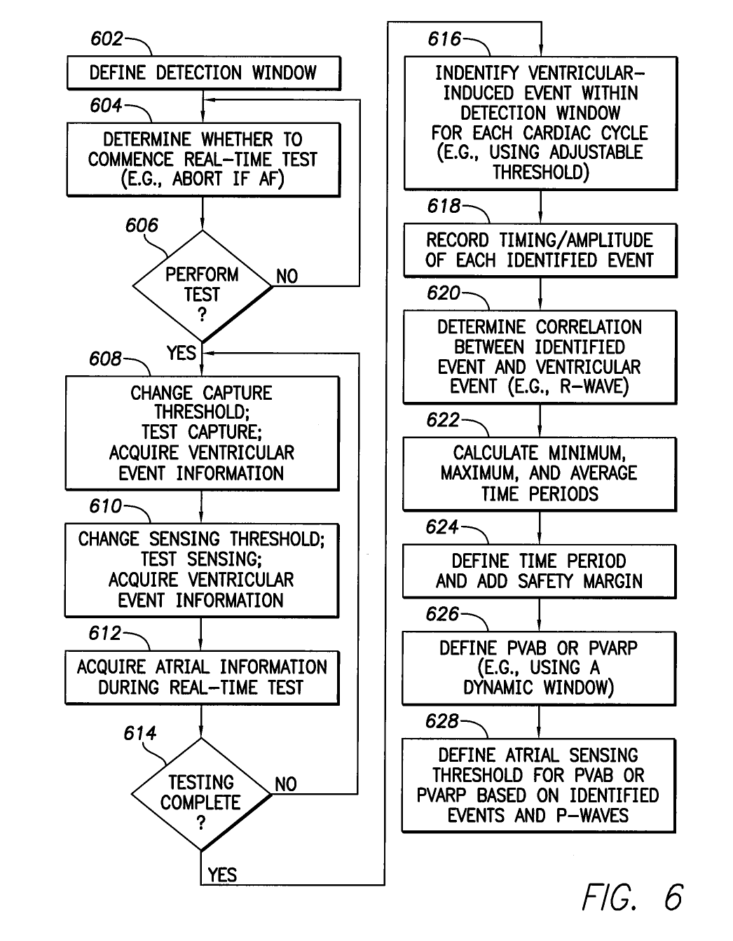

With the above in mind, additional details relating to operations that may be performed in conjunction with defining a time period in accordance with the teachings herein will be described with reference to FIG. 6. For illustration purposes, the example of FIG. 6 discusses the definition of PVAB and PVARP parameters. It should be appreciated, however, that the teachings herein may be applicable to the definition of other types of time periods.

As represented by block 602, at some point in time a detection window is defined for use in conjunction with the acquisition of data as mentioned above. In some cases this parameter may be predefined and programmed into the device 100.

In some aspects, the definition of the detection window may depend on whether it is to be used with an intrinsic ventricular depolarization event or with ventricular depolarization that is the result of a pacing operation. For example, for a paced ventricular event (e.g., a case where the ventricular depolarization results from a pacing pulse), a detection window may be defined to commence at the time of the paced event (e.g., when the pacing pulse is generated or when the resulting depolarization is sensed). This detection window may then end a defined period of time (e.g., 350 ms) after the paced event.

In contrast, for cases where the ventricular depolarization is an intrinsic event (e.g., a sensed R-wave), a detection window may be defined to commence a defined period of time (e.g., 40 ms) before an R-wave is sensed and end a defined period of time (e.g., 300 ms) after the R-wave is sensed. By defining this detection widow to cover a period of time before the R-wave is sensed, any portion of the R-wave that occurred before the time that the R-wave was sensed may be collected. As a result, the entirety of the R-wave may be made available for use in the correlation operations described below.

Turning now to blocks 604 and 606, as mentioned above, at various points in time a decision may be made to commence a real-time test. For example, a treating physician may conduct such a test upon implant to set the initial thresholds to be used by the implanted device 100. Alternatively, such a test may be performed during periodic clinical check-ups where the treating physician or a clinician may, for example, review the prior operation of the implanted device 100 and, if deemed necessary, adjust one or more operating parameters of the implanted device 100.

In some aspects, the decision to commence a real-time test may involve determining whether the test should be performed in view of current conditions. For example, in the event the patient is currently experience an episode of atrial fibrillation or some other arrhythmia, the device may abort the real-time test at block 606.

If a decision is made to perform a real-time test at block 606, the real-time test controller 302 may commence one or more tests such as a ventricular capture threshold test, a ventricular sensing threshold test, or some other type of test. In some aspects, such a test may be configured to sense various types of ventricular activity including, for example, paced ventricular activity, sensed ventricular conduction, or premature ventricular contraction. Consequently, a test also may be configured to determine the timing of any ventricular events (e.g., activity) that occurs during the test.

For example, as represented by block 608, a ventricular capture threshold test may involve controlling a cardiac pacing circuit 304 to adjust the amplitude of a ventricular pacing pulse. In this way, the implanted device 100 may determine, in real-time, the threshold level at which a pacing pulse induces depolarization of a ventricle. Moreover, whenever tissue capture is achieved in the ventricle (e.g., as detected by the cardiac sensing circuit 306 when sensing in a ventricle channel), this test may be configured to record information relating to the ventricular depolarization (e.g., the timing of the event). This information (e.g., stored as a portion of the acquired data 310) may then be used during time period definition operations (e.g., as discussed below).

As another example, as represented by block 610, a ventricular sensing threshold test may involve setting a threshold used by the cardiac sensing circuit 306 to sense a ventricular event (e.g., depolarization). Here, the sensing threshold may be adjusted (e.g., increased or decreased) to determine, in real-time, the minimum threshold level at which depolarization is detected. Again, this test may be configured to record information (e.g., timing information) relating to each detected ventricular depolarization for use in time period definition operations.

At block 612, the implanted device 100 may be configured to acquire atrial sense data during the real-time test as discussed above. For example, when the test is commenced, atrial sense amplifiers of the cardiac sensing circuit 306 may be enabled and any sensed signals processed by the signal processor 308 to provide acquired atrial data (e.g., stored as a portion of the acquired data 310).

As represented by block 614, a real-time test is generally performed over several cardiac cycles to test the effect of the different threshold levels. Consequently, the acquired cardiac data 310 may include atrial and ventricular data for several cardiac cycles. In some implementations, once the test is completed, the implanted device 100 may process the acquired cardiac data 310 to define one or more time periods as described at blocks 616-626. It should be appreciated that in some implementations one or more of these operations may be performed during the real-time test.

As represented by block 616, the cardiac event detector 314 applies the appropriate detection window to the acquired cardiac data 310 for each cardiac cycle and identifies any ventricular-induced events (e.g., far-field and/or retrograde conduction events) that occur during these time intervals. Here, the cardiac event detector 314 may distinguish far-field signals (e.g., a far-field R-wave or T-wave) or a retrograde P-wave observed in the atrial channel from, for example, a normal P-wave, a premature atrial contraction, or some other event. To this end, the cardiac event detector 314 may perform appropriate filtering, threshold detection, morphology discrimination, or some combination of these or other operations on the atrial channel data.

In some cases, the implanted device 100 may employ a software adjustable threshold to identify an event. For example, the cardiac event detector 314 may process the acquired data 310 by varying the threshold to identify far-field or retrograde events (e.g., based on programmed or learned threshold levels that correspond to different types of far-field or retrograde events). The system also may employ morphology matching for further event classification.

As represented by block 618, the implanted device 100 may record information relating to each detected event. This information may include, for example, the timing and amplitude of each event (e.g., corresponding to far-field R-waves and T-waves or to retrograde conduction).

As represented by block 620, the implanted device 100 determines the correlation between each type of ventricular-induced event and the associated ventricular event. For example, for a sensed ventricular event, the time period definer 318 may determine the timing between the sensed R-wave and the far-field R-wave for each cardiac cycle. In addition, the time period definer 318 may determine the timing between the sensed R-wave and the far-field T-wave for each cardiac cycle. For a paced ventricular event, the time period definer 318 may determine, for each cardiac cycle, the timing between the paced ventricular event and the far-field R-wave and far-field T-wave. Similarly, the time period definer 318 may determine, for each cardiac cycle, the timing between a ventricular event and retrograde conduction.

In some aspects, the determined correlation may relate to the amplitude of the respective signals. For example, an increase or decrease in the amplitude of a ventricular event may lead to a corresponding increase or decrease in the amplitude of a resulting far-field or retrograde event.

In each of the cases described above, the time period definer 318 may disqualify any events that lie outside a given timing and/or amplitude tolerance range. However, the correlation operation may take into account variations that occur normally. For example, the timing of an event may vary depending on the rate and/or amplitude of the ventricular events. Timing also may vary for different types of ventricular activity such as conducted ventricular activity, paced ventricular activity, and premature ventricular activity.

As represented by block 622, the time period definer 318 calculates one or more time period values for each event type. For example, the time period definer 318 may calculate one or more of an average, maximum, or minimum timing value for each far-field or retrograde event. In addition, the time period definer 318 may calculate one or more of an average, maximum, or minimum amplitude value for each far-field or retrograde event. Here, the timing values for each far-field or retrograde event may be relative to the timing of the associated ventricular event (e.g., R-wave).

As represented by block 624, the time period definer 318 may define one or more post-ventricular atrial time periods based on the time period values obtained at block 622. For example, the time period definer 318 may define PVAB based on the maximum time measured between ventricular depolarization and the far-field R-wave or far-field T-wave that resulted from that ventricular depolarization. Similarly, the time period definer 318 may define PVARP based on the maximum time measured between ventricular depolarization and the retrograde conduction event that resulted from that ventricular depolarization.

The time period definer 318 may then add a safety margin (e.g., a 20 ms offset) to the resulting value to provide the time period value (e.g., a proposed time period). For example, a safety margin may be added to the maximum time for a far-field R-wave or T-wave to provide a proposed time period for PVAB. Similarly, a safety margin may be added to the maximum time for a retrograde conduction event to provide a proposed time period for PVARP.

As represented by block 626, the time period definer 318 may then define the final value for each post-ventricular atrial time period (e.g., PVAB and/or PVARP). In some cases this may simply involve selecting the value(s) calculated at block 624. In other cases, this operation may be based on input from a user as discussed above in conjunction with FIG. 2.

In some cases, a time period may be defined as a dynamic window. For example, instead of blanking for the entire PVAB time period, blanking may be applied during a window of time that is defined based on one or more of the values obtained above. For example, a PVAB window for blanking atrial sensing may commence at the minimum far-field R-wave time calculated at block 622 minus an offset (e.g., 20 ms). This PVAB window may then end at the maximum far-field R-wave time calculated at block 622 plus an offset (e.g., 20 ms). The PVAB window for blanking atrial sensing may then start again at the minimum far-field T-wave time calculated at block 622 minus an offset (e.g., 20 ms). This PVAB window may then end at the maximum far-field T-wave time calculated at block 622 plus an offset (e.g., 20 ms). Also, a PVARP window for conditional atrial sensing may commence at the minimum retrograde conduction event time calculated at block 622 minus an offset (e.g., 20 ms). This PVARP window may then end at the maximum retrograde conduction event time calculated at block 622 plus an offset (e.g., 20 ms).

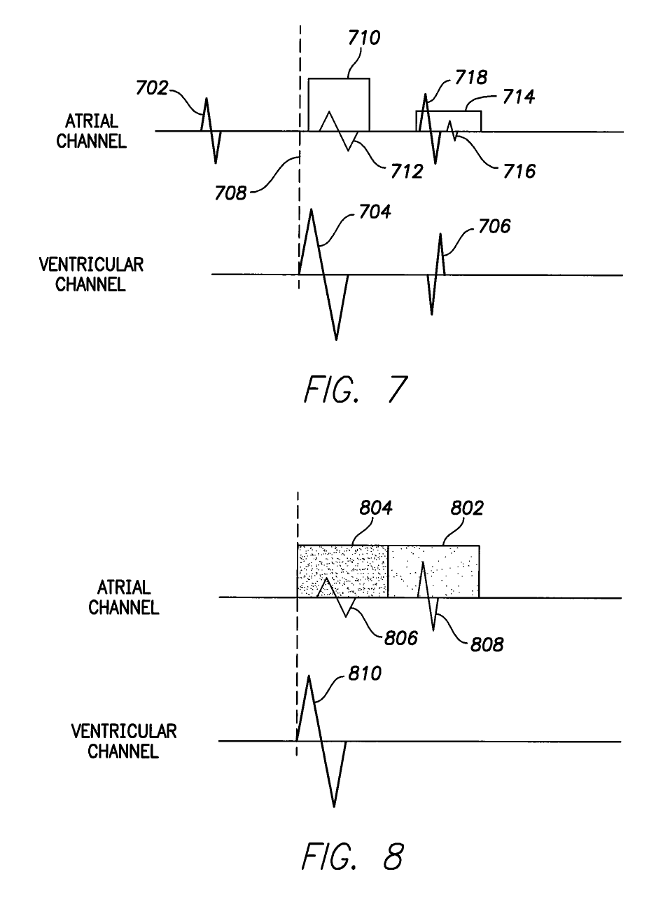

Examples of such dynamic windows are illustrated in the simplified waveform diagrams of FIGS. 7 and 8. Here, each top graph represents an atrial channel while each bottom graph represents a ventricular channel.

Referring initially to FIG. 7, a P-wave 702 in the atrial channel results in an R-wave 704 and associated T-wave 706 in the ventricular channel. Detection of the R-wave in the ventricular channel is represented by a dashed line 708.

In this example, a PVAB time period 710 does not start with the detection of the R-wave 704. Rather, the PVAB time period 710 consists of a window that commences and ends based on, for example, the minimum and maximum far-field R-wave time values described above. Here, a far-field R-wave 712 falling within the window 710 may be ignored for purposes of atrial sensing by the implanted device 100. Similarly, a PVAB time period 714 may consist of a window that commences and ends based on, for example, the minimum and maximum far-field T-wave time values described above. Here, a signal 716 (e.g., a far-field T-wave) falling within the window 714 may be ignored for purposes of atrial sensing by the implanted device 100.

Advantageously, additional atrial detection time may be provided before and after the windows 710 and 714 due to the dynamic definitions of the windows 710 and 714. That is, the implanted device 100 may detect atrial events (e.g., events associated with atrial fibrillation) during the time periods immediately preceding and following the windows 710 and 714 in contrast with PVAB schemes that define a longer and/or a continuous blanking period. Consequently, the use of a dynamic window may provide improved atrial sensing sensitivity as compared to some other PVAB schemes.

Similarly, as shown in FIG. 8, a PVARP time period 802 (e.g., for analyzing retrograde events) that follows a PVAB time period 804 (e.g., for blanking a far-field signal 806) may consist of a window that commences and ends based on, for example, the minimum and maximum retrograde conduction time values described above. Here, a signal 808 caused by a ventricular event 810 (e.g., a retrograde conduction event resulting from an R-wave) falling within the window 802 may not automatically trigger an atrial sense indication. Rather, the signal 808 may be conditionally characterized then analyzed (e.g., using morphology discrimination) to provide a more accurate characterization of the signal 808, and a different response from the device 100 may be provided accordingly.

Also, in a similar manner as the PVAB case, additional atrial detection time (not shown in FIG. 8) may be provided before and after the window 802 by dynamically defining the window 802. As a result, it may be possible to directly detect events (e.g., events associated with atrial fibrillation, a premature atrial contraction, and so on) during these preceding and following time periods, thereby improving the sensitivity of the atrial sensing as compared to PVARP schemes that define a longer refractory period.

As represented by block 628 of FIG. 6, in some implementations the implanted device 100 may define a sensing threshold that is associated with one or more of the time periods defined at block 626. Such a threshold may be used, for example, to facilitate detection of certain atrial signals (e.g., P-waves) while rejecting or conditionally classifying other signals (e.g., far-field signals). For example, if the magnitude of an event falls below a threshold level, the implanted device 100 may elect to ignore the event instead of analyzing the morphology of the event to further characterize the event.

Referring again to FIG. 7, an atrial sensing threshold may be defined corresponding to the illustrated height of a signal expected to be received during the PVAB time period 714. For example, the sensing threshold may be defined at a level such that the amplitude of a true atrial activity signal 718 (e.g., a P-wave) will be higher than the sensing threshold while the amplitude of a different type of signal 716 (e.g., a far-field T-wave) will fall below the threshold. Thus, in this case, the signal 718 may be readily detected as a sinus P-wave since it exceeds the threshold while the signal 716 is rejected (e.g., subject to blanking).

In some implementations, a similar sensing threshold may be defined for the PVARP time period as well. For example, such a sensing threshold may be defined at a level such that the amplitude of a true atrial activity signal (e.g., a P-wave) will be higher than the sensing threshold while the amplitude of a different type of signal (e.g., a retrograde event) will fall below the threshold. Thus, in this case, a signal exceeding the threshold may be readily detected as a sinus P-wave while a signal below the threshold may be further analyzed to determine whether it is a retrograde event or some other type of event.

In some aspects, the sensing threshold may be defined based on characteristics of the far-field and/or retrograde signals acquired during the real-time test. Referring to FIGS. 3 and 6, the cardiac event detector 314 may define a sensing threshold 324 based on the far-field or retrograde amplitude information generated at block 620. For example, an atrial sensing threshold may be defined as the maximum far-field or retrograde signal amplitude calculated at block 622 plus a safety margin.

The sensing threshold level also may be based on the amplitude of true atrial signals (e.g., P-waves) detected in the atrial channel. For example, the sensing threshold may be constrained to be below a minimum amplitude (including a safety margin) that has been measured for these true atrial signals.

It should be appreciated that the teachings herein may be implemented in various ways. For example, the time period definer 318 may define a time period at some time other than during a real-time test. Here, the implanted device 100 may occasionally (e.g., periodically) acquire cardiac data during normal sinus rhythm or during pacing operations. In this case, the time period definer 318 may define a time period based on the ventricular event information (e.g., R-waves) and atrial event information (e.g., far-field R-waves) acquired during these other times.

Here, a time period determination procedure may be invoked when a ventricular event is detected or is to occur (e.g., for a paced event). For example, for a paced event or premature ventricular contraction, a PVAB time period may be set to correspond to the time period measured from the ventricular event to the far-field R-wave and/or the far-field T-wave, plus an offset such as 20 ms. For a conducted R-wave, a PVAB time period may be set to the time period measured from the sensed R-wave to the far-field R-wave, plus an offset such as 20 ms. A PVARP may be determined in a similar manner based on sensed retrograde conduction events. The implanted device 100 may then proceed to use this new time period for its cardiac therapy operations.

Alternatively, the implanted device 100 may present this new time period to a user via an appropriate external device (e.g., the external device 104) at the next opportunity (e.g., when the patient moves within range of a bedside monitor, a clinic-based monitor, or some other device). Here, the user may select the time period to be used by the implanted device 100 in a manner as taught herein.

In another variation of the examples set forth above, in some implementations (e.g., for dual-chamber implantable devices) a real-time test may involve determining an atrial threshold. In such a case, certain aspects of the operations of FIG. 6 relating to acquiring cardiac data may be performed in a different way. For example, in conjunction with an atrial capture test (corresponding to block 608) or an atrial sensing test (corresponding to block 610), the implanted device 100 also may be configured to sense in a ventricular channel to acquire ventricular sense information (corresponding to block 612). Thus, in this case, the implanted device 100 may acquire ventricle information (e.g., the timing of ventricular depolarization) during the real-time atrial test.

During an atrial capture test or an atrial sensing test, however, the implanted device 100 may be either sensing or pacing a ventricle at a given point in time depending on the current system settings. That is, when performing an atrial test, either paced or sensed events, but not both, may be present. Consequently, the ventricle information acquired here may not be as deterministic as in the case of the ventricular capture sensing tests discussed above.

Accordingly, one or more system settings may be adjusted when performing an atrial capture threshold test to facilitate acquiring information that may be needed for defining an atrial time period. For example, the A-V delay may be extended to a sufficient degree when pacing the atrium to prevent the implanted device 100 from pacing a ventricle. In this way, the device 100 may be more likely to sense an R-wave that may be used to define a time period associated with this event. In addition, when atrial capture is lost during the test, the next atrial sensed events after the ventricular response, either paced or sensed, may be evaluated to determine whether they are far-field signals or retrograde events.

Similarly, one or more system settings may be adjusted when performing an atrial sensing threshold test to facilitate acquiring information that may be needed for defining an atrial time period. For example, the P-V delay may be shortened to a sufficient degree when sensing the atrium in an attempt to ensure that the implanted device 100 paces a ventricle. In this way, the device 100 may be more likely to acquire paced ventricular event information that may be used to define a time period associated with this event.

From the above it should be appreciated that a user may save time by using an implanted device that acquires atrial and ventricular information and defines time periods in conjunction with real-time tests as taught herein. For example, a physician or any other user that programs the implanted device 100 may save time that would otherwise be spent conducting multiple searches and programming procedures.

Moreover, through the use of the teachings herein, any discomfort experienced by the patient may be reduced and the patient's quality of life improved because events may be characterized with better sensitivity and more specificity. For example, using these techniques more appropriate time period values may be established upon implant. In addition, the likelihood of administration of improper treatment (e.g., improper mode switching) may be reduced since the events may be characterized with more accuracy.

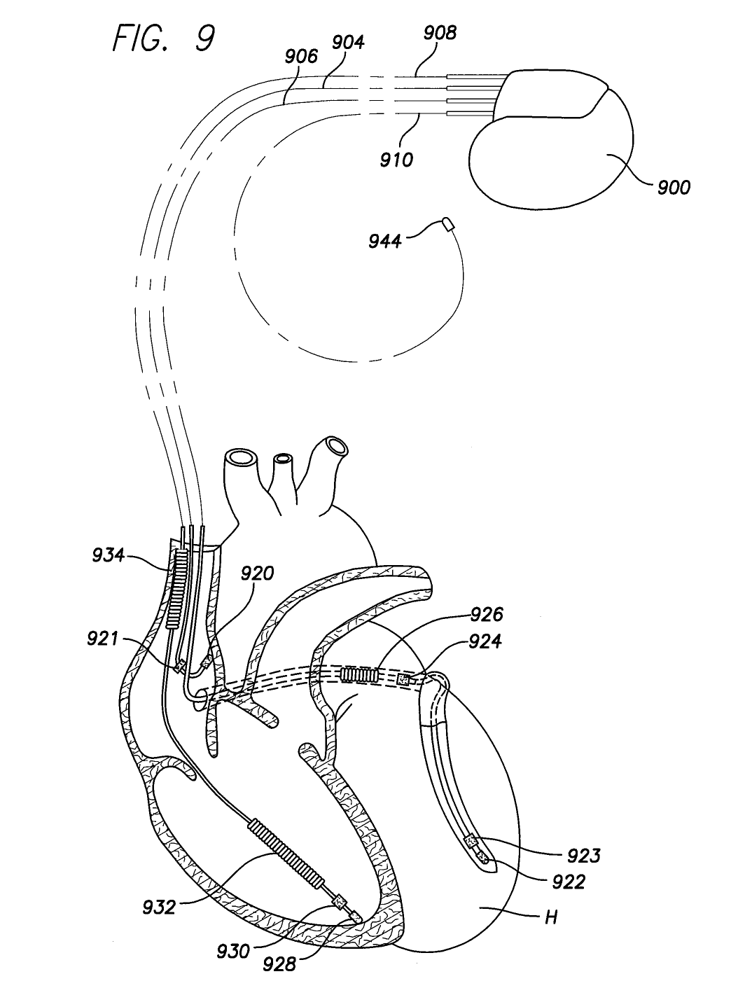

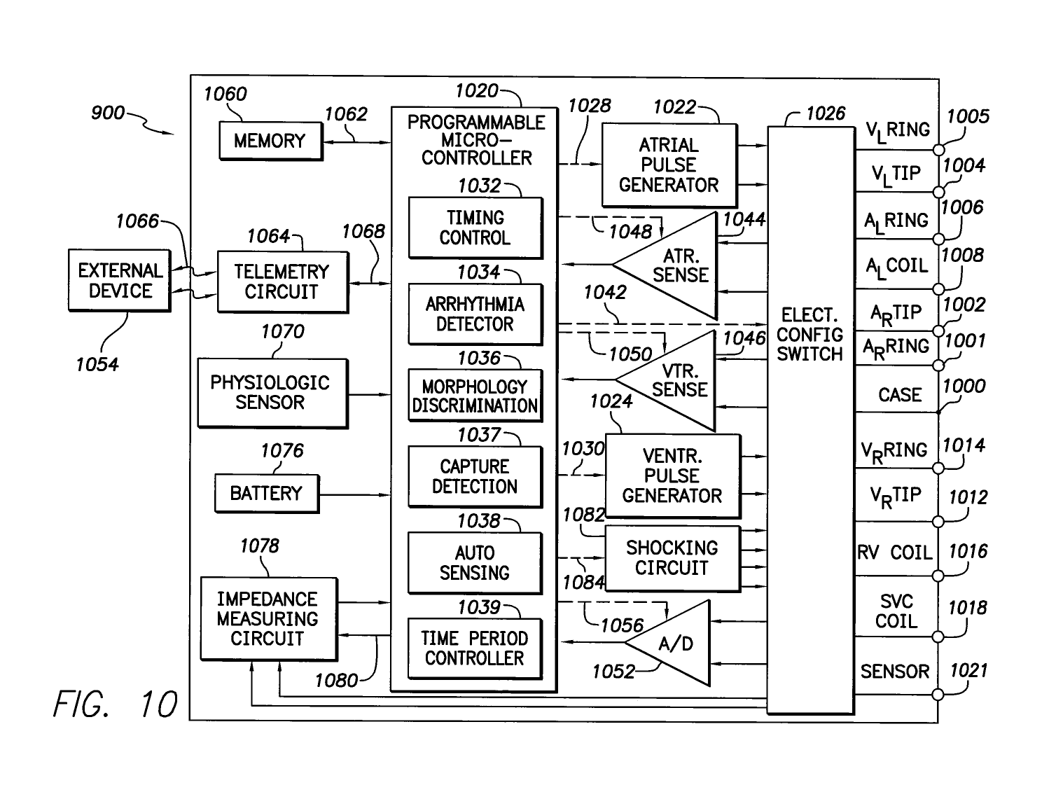

Additional details relating to monitoring, pacing, and other operations that may be performed by the implantable device 100 will now be described in the context of an implantable cardiac device as shown in FIGS. 9 and 10 (e.g., a stimulation device such as an implantable cardioverter defibrillator, a pacemaker, etc.). It is to be appreciated and understood that other cardiac devices, including those that are not necessarily implantable, may be used and that the description below is given, in its specific context, to assist the reader in understanding, with more clarity, the embodiments described herein.

FIG. 9 shows an exemplary implantable cardiac device 900 in electrical communication with a patient's heart H by way of three leads 904, 906, and 908, suitable for delivering multi-chamber stimulation and shock therapy. To sense atrial cardiac signals and to provide right atrial chamber stimulation therapy, the device 900 is coupled to an implantable right atrial lead 904 having, for example, an atrial tip electrode 920, which typically is implanted in the patient's right atrial appendage or septum. FIG. 9 also shows the right atrial lead 904 as having an optional atrial ring electrode 921.

To sense left atrial and ventricular cardiac signals and to provide left chamber pacing therapy, the device 900 is coupled to a coronary sinus lead 906 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 906 is designed to receive atrial and ventricular cardiac signals and to deliver left ventricular pacing therapy using, for example, a left ventricular tip electrode 922 and, optionally, a left ventricular ring electrode 923; provide left atrial pacing therapy using, for example, a left atrial ring electrode 924; and provide shocking therapy using, for example, a left atrial coil electrode 926 (or other electrode capable of delivering a shock). For a more detailed description of a coronary sinus lead, the reader is directed to U.S. Pat. No. 5,466,254, “Coronary Sinus Lead with Atrial Sensing Capability” (Helland), which is incorporated herein by reference.

The device 900 is also shown in electrical communication with the patient's heart H by way of an implantable right ventricular lead 908 having, in this implementation, a right ventricular tip electrode 928, a right ventricular ring electrode 930, a right ventricular (RV) coil electrode 932 (or other electrode capable of delivering a shock), and a superior vena cava (SVC) coil electrode 934 (or other electrode capable of delivering a shock). Typically, the right ventricular lead 908 is transvenously inserted into the heart H to place the right ventricular tip electrode 928 in the right ventricular apex so that the RV coil electrode 932 will be positioned in the right ventricle and the SVC coil electrode 934 will be positioned in the superior vena cava. Accordingly, the right ventricular lead 908 is capable of sensing or receiving cardiac signals, and delivering stimulation in the form of pacing and shock therapy to the right ventricle.

The device 900 is also shown in electrical communication with a lead 910 including one or more components 944 such as a physiologic sensor. The component 944 may be positioned in, near or remote from the heart.

It should be appreciated that the device 900 may connect to leads other than those specifically shown. In addition, the leads connected to the device 900 may include components other than those specifically shown. For example, a lead may include other types of electrodes, sensors or devices that serve to otherwise interact with a patient or the surroundings.

FIG. 10 depicts an exemplary, simplified block diagram illustrating sample components of the device 900. The device 900 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

Housing 1000 for the device 900 is often referred to as the “can”, “case” or “case electrode”, and may be programmably selected to act as the return electrode for all “unipolar” modes. The housing 1000 may further be used as a return electrode alone or in combination with one or more of the coil electrodes 926, 932 and 934 for shocking purposes. Housing 1000 further includes a connector (not shown) having a plurality of terminals 1001, 1002, 1004, 1005, 1006, 1008, 1012, 1014, 1016 and 1018 (shown schematically and, for convenience, the names of the electrodes to which they are connected are shown next to the terminals). The connector may be configured to include various other terminals (e.g., terminal 1021 coupled to a sensor or some other component) depending on the requirements of a given application.

To achieve right atrial sensing and pacing, the connector includes, for example, a right atrial tip terminal (AR TIP) 1002 adapted for connection to the right atrial tip electrode 920. A right atrial ring terminal (AR RING) 1001 may also be included and adapted for connection to the right atrial ring electrode 921. To achieve left chamber sensing, pacing, and shocking, the connector includes, for example, a left ventricular tip terminal (VL TIP) 1004, a left ventricular ring terminal (VL RING) 1005, a left atrial ring terminal (AL RING) 1006, and a left atrial shocking terminal (AL COIL) 1008, which are adapted for connection to the left ventricular tip electrode 922, the left ventricular ring electrode 923, the left atrial ring electrode 924, and the left atrial coil electrode 926, respectively.

To support right chamber sensing, pacing, and shocking, the connector further includes a right ventricular tip terminal (VR TIP) 1012, a right ventricular ring terminal (VR RING) 1014, a right ventricular shocking terminal (RV COIL) 1016, and a superior vena cava shocking terminal (SVC COIL) 1018, which are adapted for connection to the right ventricular tip electrode 928, the right ventricular ring electrode 930, the RV coil electrode 932, and the SVC coil electrode 934, respectively.

At the core of the device 900 is a programmable microcontroller 1020 that controls the various modes of stimulation therapy. As is well known in the art, microcontroller 1020 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 1020 includes the ability to process or monitor input signals (data or information) as controlled by a program code stored in a designated block of memory. The type of microcontroller is not critical to the described implementations. Rather, any suitable microcontroller 1020 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander et al.) and U.S. Pat. No. 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 10 also shows an atrial pulse generator 1022 and a ventricular pulse generator 1024 that generate pacing stimulation pulses for delivery by the right atrial lead 904, the coronary sinus lead 906, the right ventricular lead 908, or some combination of these leads via an electrode configuration switch 1026. It is understood that in order to provide stimulation therapy in each of the four chambers of the heart, the atrial and ventricular pulse generators 1022 and 1024 may include dedicated, independent pulse generators, multiplexed pulse generators, or shared pulse generators. The pulse generators 1022 and 1024 are controlled by the microcontroller 1020 via appropriate control signals 1028 and 1030, respectively, to trigger or inhibit the stimulation pulses.

Microcontroller 1020 further includes timing control circuitry 1032 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 1020 further includes an arrhythmia detector 1034. The arrhythmia detector 1034 may be utilized by the device 900 for determining desirable times to administer various therapies. The arrhythmia detector 1034 may be implemented, for example, in hardware as part of the microcontroller 1020, or as software/firmware instructions programmed into the device 900 and executed on the microcontroller 1020 during certain modes of operation.

Microcontroller 1020 may include a morphology discrimination module 1036, a capture detection module 1037 and an auto sensing module 1038. These modules are optionally used to implement various exemplary recognition algorithms or methods. The aforementioned components may be implemented, for example, in hardware as part of the microcontroller 1020, or as software/firmware instructions programmed into the device 900 and executed on the microcontroller 1020 during certain modes of operation.

The electrode configuration switch 1026 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 1026, in response to a control signal 1042 from the microcontroller 1020, may be used to determine the polarity of the stimulation pulses (e.g., unipolar, bipolar, combipolar, etc.) by selectively closing the appropriate combination of switches (not shown) as is known in the art.

Atrial sensing circuits (ATR. SENSE) 1044 and ventricular sensing circuits (VTR. SENSE) 1046 may also be selectively coupled to the right atrial lead 904, coronary sinus lead 906, and the right ventricular lead 908, through the switch 1026 for detecting the presence of cardiac activity in each of the four chambers of the heart. Accordingly, the atrial and ventricular sensing circuits 1044 and 1046 may include dedicated sense amplifiers, multiplexed amplifiers, or shared amplifiers. Switch 1026 determines the “sensing polarity” of the cardiac signal by selectively closing the appropriate switches, as is also known in the art. In this way, the clinician may program the sensing polarity independent of the stimulation polarity. The sensing circuits (e.g., circuits 1044 and 1046) are optionally capable of obtaining information indicative of tissue capture.

Each sensing circuit 1044 and 1046 preferably employs one or more low power, precision amplifiers with programmable gain, automatic gain control, bandpass filtering, a threshold detection circuit, or some combination of these components, to selectively sense the cardiac signal of interest. The automatic gain control enables the device 900 to deal effectively with the difficult problem of sensing the low amplitude signal characteristics of atrial or ventricular fibrillation.

The outputs of the atrial and ventricular sensing circuits 1044 and 1046 are connected to the microcontroller 1020, which, in turn, is able to trigger or inhibit the atrial and ventricular pulse generators 1022 and 1024, respectively, in a demand fashion in response to the absence or presence of cardiac activity in the appropriate chambers of the heart. Furthermore, as described herein, the microcontroller 1020 is also capable of analyzing information output from the sensing circuits 1044 and 1046, a data acquisition system 1052, or both. This information may be used to determine or detect whether and to what degree tissue capture has occurred and to program a pulse, or pulses, in response to such determinations. The sensing circuits 1044 and 1046, in turn, receive control signals over signal lines 1048 and 1050, respectively, from the microcontroller 1020 for purposes of controlling the gain, threshold, polarization charge removal circuitry (not shown), and the timing of any blocking circuitry (not shown) coupled to the inputs of the sensing circuits 1044 and 1046 as is known in the art.

For arrhythmia detection, the device 900 utilizes the atrial and ventricular sensing circuits 1044 and 1046 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 1034 of the microcontroller 1020 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (A/D) data acquisition system 1052. The data acquisition system 1052 is configured (e.g., via signal line 1056) to acquire intracardiac electrogram (“IEGM”) signals or other signals, convert the raw analog data into a digital signal, and store the digital signals for later processing, for telemetric transmission to an external device 1054, or both. For example, the data acquisition system 1052 may be coupled to the right atrial lead 904, the coronary sinus lead 906, the right ventricular lead 908 and other leads through the switch 1026 to sample cardiac signals across any pair of desired electrodes.

The data acquisition system 1052 also may be coupled to receive signals from other input devices. For example, the data acquisition system 1052 may sample signals from a physiologic sensor 1070 or other components shown in FIG. 10 (connections not shown).

The microcontroller 1020 is further coupled to a memory 1060 by a suitable data/address bus 1062, wherein the programmable operating parameters used by the microcontroller 1020 are stored and modified, as required, in order to customize the operation of the device 900 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 1052), which data may then be used for subsequent analysis to guide the programming of the device 900.

Advantageously, the operating parameters of the implantable device 900 may be non-invasively programmed into the memory 1060 through a telemetry circuit 1064 in telemetric communication via communication link 1066 with the external device 1054, such as a programmer, transtelephonic transceiver, a diagnostic system analyzer or some other device. The microcontroller 1020 activates the telemetry circuit 1064 with a control signal (e.g., via bus 1068). The telemetry circuit 1064 advantageously allows intracardiac electrograms and status information relating to the operation of the device 900 (as contained in the microcontroller 1020 or memory 1060) to be sent to the external device 1054 through an established communication link 1066.

The device 900 can further include one or more physiologic sensors 1070. In some embodiments the device 900 may include a “rate-responsive” sensor that may provide, for example, information to aid in adjustment of pacing stimulation rate according to the exercise state of the patient. One or more physiologic sensors 1070 (e.g., a pressure sensor) may further be used to detect changes in cardiac output, changes in the physiological condition of the heart, or diurnal changes in activity (e.g., detecting sleep and wake states). Accordingly, the microcontroller 1020 responds by adjusting the various pacing parameters (such as rate, A-V Delay, V-V Delay, etc.) at which the atrial and ventricular pulse generators 1022 and 1024 generate stimulation pulses.

While shown as being included within the device 900, it is to be understood that a physiologic sensor 1070 may also be external to the device 900, yet still be implanted within or carried by the patient. Examples of physiologic sensors that may be implemented in conjunction with the device 900 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 1070 may optionally include one or more of components to help detect movement (via, e.g., a position sensor or an accelerometer) and minute ventilation (via an MV sensor) in the patient. Signals generated by the position sensor and MV sensor may be passed to the microcontroller 1020 for analysis in determining whether to adjust the pacing rate, etc. The microcontroller 1020 may thus monitor the signals for indications of the patient's position and activity status, such as whether the patient is climbing up stairs or descending down stairs or whether the patient is sitting up after lying down.

The device 900 additionally includes a battery 1076 that provides operating power to all of the circuits shown in FIG. 10. For a device 900 which employs shocking therapy, the battery 1076 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 1076 also desirably has a predictable discharge characteristic so that elective replacement time can be detected. Accordingly, the device 900 preferably employs lithium or other suitable battery technology.

The device 900 can further include magnet detection circuitry (not shown), coupled to the microcontroller 1020, to detect when a magnet is placed over the device 900. A magnet may be used by a clinician to perform various test functions of the device 900 and to signal the microcontroller 1020 that the external device 1054 is in place to receive data from or transmit data to the microcontroller 1020 through the telemetry circuit 1064.

The device 900 further includes an impedance measuring circuit 1078 that is enabled by the microcontroller 1020 via a control signal 1080. The known uses for an impedance measuring circuit 1078 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 900 has been implanted; measuring stroke volume; and detecting the opening of heart valves, etc. The impedance measuring circuit 1078 is advantageously coupled to the switch 1026 so that any desired electrode may be used.

In the case where the device 900 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 1020 further controls a shocking circuit 1082 by way of a control signal 1084. The shocking circuit 1082 generates shocking pulses of low (e.g., up to 0.5 J), moderate (e.g., 0.5 J to 10 J), or high energy (e.g., 11 J to 40 J), as controlled by the microcontroller 1020. Such shocking pulses are applied to the patient's heart H through, for example, two shocking electrodes and as shown in this embodiment, selected from the left atrial coil electrode 926, the RV coil electrode 932 and the SVC coil electrode 934. As noted above, the housing 1000 may act as an active electrode in combination with the RV coil electrode 932, as part of a split electrical vector using the SVC coil electrode 934 or the left atrial coil electrode 926 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 1020 is capable of controlling the synchronous or asynchronous delivery of the shocking pulses.

As mentioned above, the device 900 may include several components that provide functionality relating to defining time periods as taught herein. For example, one or more of the switch 1026, the sensing circuits 1044, 1046, and the data acquisition system 1052 may acquire cardiac signals that are used in the time period definition operations discussed above, with reference to FIGS. 2 and 6. In addition, the data described above may be stored in the memory 1060.

The microcontroller 1020 (e.g., a processor providing signal processing functionality) also may implement or support at least a portion of the time period definition functionality discussed herein. For example, a time period controller 1039 may define PVAB and PVARP intervals and related thresholds as described above with reference to FIG. 6.

It should be appreciated that various modifications may be incorporated into the disclosed embodiments based on the teachings herein. For example, the structure and functionality taught herein may be incorporated into types of devices other than the specific types of devices described above. In addition, different types of time periods may be defined based on the teachings herein. Also, different types of cardiac signals may be acquired in conjunction with defining a time periods and these signals may be detected in various ways. Different techniques may be used to provide a proposed time period to a user and receive a response from a user. Also, various algorithms or techniques may be employed to determine a time period from acquired data.

It should be appreciated from the above that the various structures and functions described herein may be incorporated into a variety of apparatuses (e.g., a stimulation device, a lead, a monitoring device, etc.) and implemented in a variety of ways. Different embodiments of such an apparatus may include a variety of hardware and software processing components. In some embodiments, hardware components such as processors, controllers, state machines, logic, or some combination of these components, may be used to implement the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may send raw data or processed data to an external device that then performs the necessary processing.

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure as defined by any claims associated herewith.

What is claimed is:

1. A method of defining an atrial time period, comprising:
conducting a test to determine at least one cardiac threshold;
acquiring atrial sense data during the test;
identifying an atrial event caused by a ventricular event from a portion of the acquired data that corresponds in time to a detection window that is based on timing of a ventricular depolarization; and
defining at least one post-ventricular atrial time period based on the identified atrial event.

2. The method of claim 1, wherein the identified atrial event comprises a far-field event or a retrograde conduction event.

3. The method of claim 1, wherein the at least one post-ventricular atrial time period comprises a post-ventricular atrial blanking period and/or a post-ventricular atrial refractory period.

4. The method of claim 1, wherein the test comprises a real-time test for determining a capture threshold and/or a sensing threshold.

5. The method of claim 1, wherein the at least one cardiac threshold comprises a ventricular capture threshold and/or a ventricular sensing threshold.

6. The method of claim 1, wherein the at least one cardiac threshold comprises an atrial capture threshold and/or an atrial sensing threshold.

7. The method of claim 1, wherein defining the at least one post-ventricular atrial time period comprises:
providing a proposed post-ventricular atrial time period; and
receiving an indication from a user input device that specifies a value for the at least one post-ventricular atrial time period.

8. The method of claim 7, wherein the providing comprises displaying.

9. The method of claim 8, wherein the value is selected by a user in response to the displayed proposed post-ventricular atrial time period.

10. The method of claim 1, wherein:
the ventricular depolarization is an intrinsic event;
the detection window commences a first defined period of time before the ventricular depolarization; and
the detection window ends a second defined period of time after the ventricular depolarization.

11. The method of claim 1, further comprising generating a pacing pulse, wherein:
the generation of the pacing pulse causes the ventricular depolarization;
the detection window commences upon generation of the pacing pulse; and
the detection window ends a defined period of time after the generation of the pacing pulse.

12. The method of claim 1, wherein identifying the atrial event comprises adjusting an atrial threshold to determine timing and/or amplitude of an event defined by the acquired atrial sense data.

13. The method of claim 1, wherein identifying the at least one post-ventricular atrial time period comprises correlating timing of the identified atrial event and the timing of the ventricular depolarization.

14. The method of claim 1, wherein the definition of the at least one post-ventricular atrial time period is based on:
a maximum time that is based on timing of the identified atrial event and the timing of the ventricular depolarization,
a minimum time that is based on timing of the identified atrial event and the timing of the ventricular depolarization, or
an average time that is based on timing of the identified atrial event and the timing of the ventricular depolarization.

15. The method of claim 14, wherein the definition of the at least one post-ventricular atrial time period is further based on a safety margin.

16. The method of claim 1, wherein:
the at least one post-ventricular atrial time period comprises a first time interval and a second time interval;
the first time interval commences after commencement of the ventricular depolarization; and
the second time interval commences after the first time interval ends.

17. The method of claim 1, further comprising defining an atrial sensing threshold associated with the at least one post-ventricular atrial time period, wherein the definition of the atrial sensing threshold is based on an amplitude of the identified atrial event and an amplitude of a P-wave.

18. An apparatus for defining an atrial time period, comprising:
a test controller configured to conduct a test to determine at least one cardiac threshold;
a sensing circuit configured to acquire atrial sense data during the test;
an event detector configured to identify an atrial event caused by a ventricular event from a portion of the acquired data that corresponds in time to a detection window that is based on timing of ventricular depolarization; and
time period definer configured to define at least one post-ventricular atrial time period based on the identified atrial event.

19. The apparatus of claim 18, wherein the identified atrial event comprises a far-field event or a retrograde conduction event.

20. The apparatus of claim 18, wherein the at least one post-ventricular atrial time period comprises a post-ventricular atrial blanking period and/or a post-ventricular atrial refractory period.

21. The apparatus of claim 18, wherein the test comprises a real-time test for determining a capture threshold and/or a sensing threshold.

22. The apparatus of claim 18, wherein the at least one cardiac threshold comprises a ventricular capture threshold and/or a ventricular sensing threshold.

23. The apparatus of claim 18, wherein the at least one cardiac threshold comprises an atrial capture threshold and/or an atrial sensing threshold.

24. The apparatus of claim 18, wherein the time period definer is further configured to define the at least one post-ventricular atrial time period by:
providing a proposed post-ventricular atrial time period; and
receiving an indication from a user input device that specifies a value for the at least one post-ventricular atrial time period.

25. The apparatus of claim 18, wherein the event detector is further configured to identify the atrial event by adjusting an atrial threshold to determine timing and/or amplitude of an event defined by the acquired atrial sense data.
